(19) United States
(12) Patent Application Publication
(10) Pub. No.: US 2012/0197044 A1
(43) Pub. Date: Aug. 2, 2012

(54) PRODUCTION METHOD FOR 4,4'-BIS(ALKYLAMINO) DIPHENYLAMINE

(75) Inventors: Jin Eok Kim, Yuseong-gu Daejeon (KR); Seung Il Kim, Yuseong-gu Daejeon (KR); Jung Hee Jang, Yuseong-gu Daejeon (KR); Han Jin Kwag, Seo-gu Daejeon (KR)

(73) Assignee: Korea Kumho Petrochemical Co., Ltd., Seoul (KR)

(21) Appl. No.: 13/502,052

(22) PCT Filed: Sep. 30, 2010
(86) PCT No.: PCT/KR2010/006674

§ 371 Date: Apr. 17, 2012

(30) Foreign Application Priority Data
Oct. 15, 2009 (KR) 10-2009-0098371

(51) Int. Cl. C07C 209/36

(52) U.S. Cl. 564/420

(57) ABSTRACT

The present invention relates to a production method for 4,4′-bis(alkylamino)diphenylamine, and more specifically relates to a method in which 4,4′-dinitrodiphenylamine (4,4′-DNDPA) is produced by reacting urea with nitrobenzene in the presence of a polar organic solvent and a base catalyst, and then the 4,4′-bis(alkylamino)diphenylamine (4,4′-BAADA) is produced by subjecting the 4,4′-DNDPA and a ketone to hydrogenation in the presence of a hydrogenation catalyst.

### TECHNICAL FIELD

The present invention relates to a production method for 4,4′-bis(alkylamino)diphenylamine.

### BACKGROUND ART

4,4′-DNDPA can be easily converted to 4,4′-diamino diphenylamine (4,4′-DADPA) and used as a raw material for an antioxidant of dyes and rubber. Japanese Patent Application Publication No. Pyo 10-168038 (1998) describes a method for synthesizing DADPA by a reduction of 4,4′-DNDPA. In the above mentioned patent publication, a 4,4′-DNDPA compound is described as being greatly effective for an intermediate of an antioxidant or anti-aging agent. As such, 4,4′-DADPA or 4,4′-DADPA derivatives are used as an intermediate of dyes, agricultural chemicals and medical substances as well as that of rubber additives.

An example of conventional methods for preparing 4,4′-DNDPA involves nitration and deacetylation of N-acetyldiphenylamine. Such a method, however, is problematic in that the nitration is not carried out uniformly. Furthermore, there is an inconvenience in that nitrodiphenylamine inevitably produced should be removed using a recrystallization from alcohol. With another method for preparing 4,4′-DNDPA, U.S. Pat. No. 4,990,673 discloses a method in which 4-chloroaniline is reacted with alkali metal cyanate to produce 4,4′-DNDPA. However, a disadvantage with the latter method is that the reaction must be carried out at 160° C. or above for a period of time longer than 15 hours.

In addition, known is another method of preparing a DNDPA derivative by the reaction of 4-nitroaniline with a nitrobenzene derivative (J. Am. Chem. Soc., 1976, (1), pp 138-143). In the above reaction, 4-nitroaniline and nitrobenzene derivatives are allowed to react using potassium t-butoxide (t-BuOK) as a base, thereby producing 2,4′-DNDPA, 4,4″-DNDPA, or 5-chloro (bromo)-2,9′-dinitrodiphenylamine as a product in which ortho-, parahydrogen or halogen is substituted, respectively, depending on the amount of the base. However, there are problems in that 4-NA must be prepared in a separate reaction and it is difficult to selectively prepare 4,4′-DNDPA.

U.K. Patent No. 1091376 discloses a method of producing 4-amino-4′-alkylamino diphenylamine by reacting DADPA with a ketone in the presence of a precious metal catalyst at high temperature and high pressure. Japanese Patent Application Publication No. Pyo 10-219243 describes a method of preparing 4,4′-BAADA by reacting 4,4′-DADPA with a ketone. However, these methods problematic in that 4,4′-DNDPA is converted to 4,4′-DADPA first, followed by reacting with a ketone, and it is difficult to purely prepare 4,4′-DADPA.

A nucleophilic aromatic substitution for hydrogen (NASH) recently proposed is a reaction in which amines or amides are directly reacted with nitrobenzene or its derivative in the presence of a base catalyst, and thus, is advantageous in that any hazardous material or any intermediate, difficult to be removed, is not produced.

A process is also known wherein aniline and nitrobenzene are directly reacted in the presence of a base, such as tetramethyl ammonium hydroxide (TMA(OH)), to prepare 4-nitrodiphenylamine (4-NDPA) and 4-nitrosodiphenylamine [J. Am. Chem. Soc., 1992, 114(23), 9237-8; U.S. Pat. No. 5,117,063; U.S. Pat. No. 5,253,737; U.S. Pat. No. 5,331,099; U.S. Pat. No. 5,453,541; U.S. Pat. No. 5,552,531; and U.S. Pat. No. 5,633,407].

In addition, the use of the NASH reaction is described in U.S. Pat. No. 5,436,371, U.S. Pat. No. 5,380,407 and International Patent Publication No. WO 93/24447. These patents disclose a method of synthesizing N-(4-nitrophenyl)benzamide using benzamide rather than aniline. More particularly, there is described a method of preparing 4-nitroaniline by synthesizing N-(4-nitrophenyl)benzamide using nitrobenzene in the amount of about 1 mole based on benzamide and hydrolyzing the resulting product with water or ammonia. However, there is no mention of the preparation of 4,4′-DNDPA. This seems to be because N-(4-nitrophenyl)benzamide produced by the reaction of amide and nitrobenzene is a stable and separable compound, so that it is no longer reacted with nitrobenzene.

### DISCLOSURE

### Technical Problem

The present inventors have therefore endeavored to overcome the problems in the prior art described above, and developed a method for producing 4,4′-BAADA with high yield and purity, which is characterized by employing the NASH reaction as mentioned above, and utilizing urea instead of aniline or benzamide. More particularly, the method of the present invention prepares 4,4′-BAADA by reacting urea with an excessive amount of nitrobenzene to selectively produce 4,4′-DNDPA and subjecting 4,4′-DNDPA and a ketone to hydrogenation in the presence of hydrogen and a hydrogenation catalyst.

The method of the present invention is advantageous in that the reaction is relatively simple while allowing the selective production of only 4,4′-DNDPA using relatively cheap raw materials without producing other by-products. Therefore, the object of the present invention is to provide a method for the production of 4,4′-BAADA with high yield and purity without employing a complicated purification process.

### Technical Solution

The present invention is characterized by providing a method for producing 4,4′-bis(alkylamino)diphenylamine, which comprises the following steps of:

1) reacting urea with nitrobenzene in the presence of a polar organic solvent and a base to prepare 4,4′-dinitrodiphenylamine; and

2) subjecting 4,4′-dinitrodiphenylamine prepared in step 1) and a ketone to hydrogenation in the presence of a hydrogenation catalyst.

### Advantageous Effects

The method of the present invention can produce 4,4′-BAADA in a simple high yield process in which 4,4′-DADPA directly reacts with a ketone, thereby not producing 4,4′-DADPA as an intermediate. Therefore, the method of the present invention is advantageous in that a selective production of only 4,4′-DNDPA can be achieved by using a relatively cheap alkali base without producing other by-products.

### BEST MODE

Hereinafter, the present invention will be described in more detail.

The present invention relates to a method for producing 4,4′-bis(alkylamino)diphenylamine (4,4′-BAADA) by reacting urea with nitrobenzene in the presence of a polar organic solvent and a base to prepare 4,4′-dinitrodiphenylamine (4,4′-DNDPA), followed by subjecting 4,4′-DNDPA and a ketone to hydrogenation in the presence of a hydrogenation catalyst.

The production method of 4,4′-BAADA according to the present invention will be described in more detail as follows.

Firstly, as a reactant, urea and nitrobenzene are reacted in the presence of a polar organic solvent and a base, to thereby prepare 4,4′-dinitrodiphenylamine. Here, considering solubility of the urea and base, it is preferable to use dimethylsulfoxide (DMSO), dimethylformamide, N-methylpyrrolidinone and the like as a polar organic solvent. Further, it is preferable to use the reactants (urea and 4-nitrobenzene) and the polar organic solvent in a volume ratio ranging from 1:1 to 1:50, more preferably 1:1 to 1:30.

In the reactants, the molar ratio of urea and nitrobenzene is preferably in the range of 1:1 to 1:16, more preferably 1:4 to 1:8 in terms of yield. The base suitable for the present invention may include akali metal bases, akaline earth metal bases and organic amine bases, and more specifically, include sodium hydroxide (NaOH), potassium hydroxide (KOH), t-potassium t-butoxide (t-BuOK), tetramethyl ammoniumhydroxide (TMA(OH)) and the like. It is preferable to use the urea and base in a molar ratio ranging from 1:1 to 1:20, more preferably 1:4 to 1:12. Where the molar ratio of the base to urea is lower than said range, there is a problem in that 4-nitroaniline remains.

The step of preparing 4,4′-dinitrodiphenylamine is preferably carried out at a temperature ranging from normal temperature (20° C.) to 100° C., more preferably 50 to 80° C. in the presence of oxygen, air or nitrogen. If the reaction temperature is too low, the reaction rate is too slow, while if it is too high, the reaction yield is remarkably decreased due to the decomposition of urea. The lower the reaction temperature, the longer the time during which 4-nitroaniline produced at the beginning of the reaction is converted into 4,4′-DNDPA. In this case, although the time required for the reaction becomes longer, the extended reaction time results in an increase in yield of a product. Where the reaction temperature exceeds the above range, the yield of the product is high at the beginning of the reaction. However, the yield of the product increases no longer from the level at the beginning of the reaction due to the decomposition of urea. Accordingly, the reaction temperature is appropriately determined within the range which is suitable to prevent urea from decomposing while increasing the reaction rate, thereby obtaining an increased yield of the product.

Moreover, where the moisture content in the reaction system is 5% or less based on the weight of the entire reaction solution, it does not have a significant effect on reactivity. Thus, no particular process to remove the moisture of the solvent is used. When the reaction is carried out in a state in which water is added to the solvent in an amount of 1%, a high yield is exhibited at the beginning of the reaction. In this case, however, the yield exhibited after the reaction of about 6 hours is not so high, as compared to that of the case where water is not added. In addition, an excess of azoxybenzene is produced under an atmosphere not containing oxygen, while no azoxybenzene is produced under an atmosphere containing oxygen.

After the reaction is completed, the base catalyst is removed by filtration, and the reaction solvent is removed by distillation, which can be recycled. In order to separate and purify 4,4′-DNDPA, water or an acidic aqueous solution is added to the reaction solution, and simultaneously, a non-polar organic solvent is added thereto. The resulting reaction solution is cooled down under reflux and solidified. The amount of water or an acidic aqueous solution used in the reaction is preferably in the range of 3- to 7-fold (weight ratio) based on the weight of the product, and that of the non-polar organic solvent is preferably in the range of 1- to 5-fold (weight ratio). Where the amount is too high, the reaction yield is remarkably decreased due to a decrease in solubility, while where it is too low, it is difficult to remove the solvent and by-products. The non-polar organic solvent suitable for the present invention may include toluene, hexane, hexane, cyclohexane and the like. Through the separation and purification process, highly pure 4,4′-DNDPA is obtained, which can be used as a raw material for preparing 4,4′-BAADA without an additional purification process.

In the step 2), 4,4′-dinitrodiphenylamine prepared in the step 1) and a ketone are subjected to hydrogenation in the presence of a hydrogenation catalyst, to thereby prepare 4,4′-bis(alkylamino)diphenylamine.

The hydrogenation catalyst suitable for the present invention may include precious metals (such as Pd and Pt) or reduction catalysts (such as Ni—Fe—Al, Cu—Cr, Raney Ni), and more preferably, support catalysts in which precious metals are supported on carbon such as Pt/C or Pd/C can be used. The hydrogenation catalyst is preferably used in the amount of 0.05 to 0.2 parts by weight based on 1 part by weight of 4,4′-DNDPA. If the amount is too low, the reaction yield is decreased, while if it is too high, there is a problem in lowering economical efficiency.

Further, the ketone suitable for the preparation of 4,4′-BAADA may include an alkylketone and a cycloalkylketone wherein the ring contains 1 to 10 carbon atoms. Examples of the alkylketone are preferably acetone, methylisobutylketone (MIBK), methylisoamylketone, methlyethylketone, methyl-n-butylketone and the like. Examples of the cycloalkylketone are preferably cyclohexanone, methylcyclohexylketone and the like. The ketone is used not only as a reactant but also as a solvent, and is preferably used in the amount of 3 to 10 mole based 1 mole of 4,4-DNDPA.

The hydrogenation of step 2) is a pressure reaction, and thus, its reaction rate can be varied depending on reaction temperature and hydrogen pressure. Generally, the hydrogenation is carried out at a reaction pressure ranging from 300 to 1000 psi and at a reaction temperature ranging from 50 to 200° C.

After the reaction is completed, the ketone as a reactant and a solvent is concentrated and removed, thereby recover 4,4′-BAADA as a final product.

It has been found that as a result of using 4,4′-BAADA prepared by the method of the present invention as an anti-aging agent for a tire rubber which is comprised of natural and synthetic rubber, it shows improvement in anti-aging effects by 10 to 30% as compared to 6PPD (N-(1,3-Dimethylbutyl)-N′-phenyl-p-phenylenediamine) which is currently on the market as an anti-aging agent.

Therefore, 4,4′-BAADA prepared according to the present invention can be effectively used as anti-aging agent for a polymer such as SBR, NBR, BR, IR, CR and EPDM.

### MODE FOR INVENTION

The foregoing and other objects, features and advantages of the invention will be apparent to those skilled in the art to which the present invention relates from reading the following specification.

### Reference Example

In the following Examples, products were analyzed by the Nuclear Magnetic Resonance (NMR) spectrum and the Gas Chromatography-Mass Spectroscopy Detector (GC-MSD) to identify them. Also, the products were quantitatively analyzed according to the following condition by high performance liquid chromatography (HPLC).

In HPLC, all quantitative values were measured at a wavelength of 254 nm, and developing speed of an eluent was 1 ml/minute. Furthermore, in HPLC, a Cosmosil 5C18-AR (4.6×150 mm) packed column was used. An elution gradient of HPLC was as follows.

Elution gradient

| Solvent | Solvent A | Solvent B |
|---|---|---|
| Time (min) | % Distilled water | % Acetonitrile |
| 0 | 65 | 35 |
| 25 | 0 | 100 |
| 33 | 65 | 35 |

For the quantitative analysis of each product, pyrene was used as an internal standard. An area ratio for a concentration of each product was calculated relative to an area of pyrene and standard-calibrated. A molar concentration of a product was calculated from the calibration curve.

### Preparation Example

### Preparation of 4,4′-DNDPA

### Preparation Example 1

Into a 2 L three-necked reactor equipped with a condenser and a thermometer were charged 30 g (0.5 mol.) of urea, 204 g (6 mol.) of sodium hydroxide and 900 g of DMSO as a solvent, followed by adding 246 g (2 mol.) of nitrobenzene drop by drop. Thereafter, the resulting solution was stirred for 30 minutes while passing oxygen, and then reacted at 60° C. The progress of the reaction was confirmed by HPLC. After the reaction was completed, the product was cooled down. Analysis of the product by HPLC showed that, where the reaction was carried out for 6 hours, 4,4′-DNDPA was obtained in a yield of 82 mol %, and where the reaction was carried out for 8 hours, 4,4′-DNDPA was obtained in a yield of 94 mol %.

Sodium hydroxide in the reaction solution was removed by using a filter. Thus filtered reaction solution was concentrated by using a vacuum distillation apparatus at 70 to 80° C. and 10 to 20 mmHg, to thereby remove unreacted nitrobenzene and DMSO. After the concentration was completed, 200 g of toluene was added to the reactor while maintaining its temperature at about 70° C., followed by stirring. After 250 g of water was added thereto, the resulting reaction solution was cooled down to solidify a product. Thus generated solids were separated by using a filter and washed with toluene. Thereafter, the solids were further purified by using toluene and water, and then, dried.

### Preparation Example 2

Reactions were carried out under the same conditions as those in Preparation Example 1 except that the reaction temperature was changed as described in Table 1. The results are shown in Table 1 below.

TABLE 1
Change in yield depending on reaction temperature

| Reaction | | Yield (mole %) | |
|---|---|---|---|
| temperature (° C.) | Time (h) | 4-NA*¹⁾ | 4,4′-DNDPA |
| 80 | 8 | — | 59 |
| 70 | 6 | — | 67 |
| 60 | 6 | — | 82 |
| | 8 | — | 94 |
| 50 | 6 | 8.9 | 55 |
| | 8 | 6.8 | 63 |

*¹⁾4-NA: 4-nitroaniline

### Preparation Example 3

Reactions were carried out under the same conditions as those in Preparation Example 1 except that the kind and amount of base were changed as described in Table 2. The results are shown in Table 2 below.

TABLE 2
Change in yield depending on the kind and amount of base

| | Base amount | Time | Yield (%) | |
|---|---|---|---|---|
| Base | g (mole) | (h) | 4-NA | 4,4′-DNDPA |
| Potassium hydroxide | 448(8) | 8 | 9 | 52 |
| Potassium t-butoxide* | 112.5(1) | 8 | — | 37 |
| Tetramethyl ammonium | 215(1.18) | 8 | — | 51 |
| hydroxide* | | | | |

*In case of potassium t-butoxide and tetramethyl ammonium hydroxide, 15 g (0.25 mol.) of urea was used

### Preparation Example 4

Reactions were carried out under the same conditions as those in Preparation Example 1 except that the amount of base was changed as described in Table 3. The results are shown in Table 3 below.

TABLE 3
Change in yield depending on the amount of base

| Base amount | Reaction | Yield (%) | |
|---|---|---|---|
| g (mole) | time (h) | 4-NA | 4,4′-DNDPA |
| 80(2) | 8 | 14 | 55 |
| 160(4) | 8 | 5 | 67 |
| 240(6) | 8 | — | 94 |
| 320(8) | 8 | — | 92 |

### Preparation Example 5

Reactions were carried out under the same conditions as those in Preparation Example 1 except that the amount of 4-nitrobenzene relative to urea was changed as described in Table 4. The results are shown in Table 4 below.

TABLE 4
Change in yield depending on the amount of 4-nitrobenzene

| NB amount | Reaction | Yield (%) | |
|---|---|---|---|
| g (mole) | time (h) | 4-NA | 4,4′-DNDPA |
| 123(1) | 8 | 7 | 67 |
| 246(2) | 8 | — | 94 |
| 492(4) | 8 | — | 75 |

### Preparation Example 6

Reactions were carried out under the same conditions as those in Preparation Example 1 except that the reaction atmosphere was changed as described in Table 5. The results are shown in Table 5 below.

TABLE 5
Change in yield depending on the reaction atmosphere

| Reaction | Reaction | Yield (%) | |
|---|---|---|---|
| atmosphere | time (h) | 4-NA | 4,4′-DNDPA |
| Oxygen | 8 | — | 94 |
| Nitrogen | 8 | 2 | 50 |
| Air | 8 | 3 | 56 |

### Example

### Preparation of 4,4′-BAADA

### Example 1

Into a high-pressure reactor were charged 259 g (1 mol.) of 4,4′-DNDPA prepared in Preparation Example 1 and 500 g (5 mol.) of MIBK (methylisobutylketone) as a reactant and solvent, followed by stirring. After 10 g (including 50% water) of 3% Pt/C as a catalyst was added thereto, the reactor was assembled and charged with nitrogen under stirring, to thereby replace indoor air of the reactor with nitrogen twice. The pressure of the reactor was adjusted to about 600 psi by injecting hydrogen thereinto, followed by increasing a reaction temperature to 160° C. The hydrogen pressure was adjusted to the range of 600 to 650 psi with maintaining the reaction temperature, and then, the reaction was carried out for 3 hours. After the reaction was completed, the reaction temperature was cooled down to 80° C., the residual hydrogen gas was discharged from the reactor, and then, the resulting reaction solution was filtered to recover the catalyst.

After thus filtered reaction solution was transferred to a concentrator, MIBK as a solvent and reactant was removed by distillation, to thereby obtain 356 g of 4,4′-bis(1,3-dimethylbutylamino)diphenylamine as a product (yield: 97%, as compared to 4,4′-DNDPA).

### Example 2

Into a high-pressure reactor were charged 259 g (1 mol.) of 4,4′-DNDPA prepared in Preparation Example 1 and 500 g (8.6 mol.) of acetone as a reactant and a solvent, followed by stirring. After 10 g (including 50% water) of 3% Pt/C as a catalyst was added thereto, the reactor was assembled and charged with nitrogen under stirring, to thereby replace indoor air of the reactor with nitrogen twice. The pressure of the reactor was adjusted to about 600 psi by injecting hydrogen thereinto, followed by increasing a reaction temperature to 160° C. The hydrogen pressure was adjusted to the range of 600 to 650 psi with maintaining the reaction temperature, and then, the reaction was carried out for 3 hours. After the reaction was completed, the reaction temperature was cooled down to 80° C., the residual hydrogen gas was discharged from the reactor, and then, the resulting reaction solution was filtered to recover the catalyst.

After thus filtered reaction solution was transferred to a concentrator, acetone as a solvent and reactant was removed by distillation, to thereby obtain 272 g of 4,4′-bis(isopropylamino)diphenylamine as a product (yield: 96%, as compared to 4,4′-DNDPA).

### Example 3

Into a high-pressure reactor were charged 259 g (1 mol.) of 4,4′-DNDPA prepared in Preparation Example 1 and 500 g (5.1 mol.) of cyclohexanone as a reactant and a solvent, followed by stirring. After 10 g (including 50% water) of 3% Pt/C as a catalyst was added thereto, the reactor was assembled and charged with nitrogen under stirring, to thereby replace indoor air of the reactor with nitrogen twice. The pressure of the reactor was adjusted to about 600 psi by injecting hydrogen thereinto, followed by increasing a reaction temperature to 160° C. The hydrogen pressure was adjusted to the range of 600 to 650 psi with maintaining the reaction temperature, and then, the reaction was carried out for 3 hours. After the reaction was completed, the reaction temperature was cooled down to 80° C., the residual hydrogen gas was discharged from the reactor, and then, the resulting reaction solution was filtered to recover the catalyst.

After thus filtered reaction solution was transferred to a concentrator, cyclohexanone as a solvent and reactant was removed by distillation, to thereby obtain 341 g of 4,4′-bis(cyclohexylamino)diphenylamine as a product (yield: 94%, as compared to 4,4′-DNDPA).

### Example 4

Into a high-pressure reactor were charged 259 g (1 mol.) of 4,4′-DNDPA prepared in Preparation Example 1, 250 g (2.5 mol.) of MIBK and 250 g (4.3 mol.) of acetone as a reactant and a solvent, followed by stirring. After 10 g (including 50% water) of 3% Pt/C as a catalyst was added thereto, the reactor was assembled and charged with nitrogen under stirring, to thereby replace indoor air of the reactor with nitrogen twice. The pressure of the reactor was adjusted to about 600 psi by injecting hydrogen thereinto, followed by increasing a reaction temperature to 160° C. The hydrogen pressure was adjusted to the range of 600 to 650 psi with maintaining the reaction temperature, and then, the reaction was carried out for 3 hours. After the reaction was completed, the reaction temperature was cooled down to 80° C., the residual hydrogen gas was discharged from the reactor, and then, the resulting reaction solution was filtered to recover the catalyst.

After thus filtered reaction solution was transferred to a concentrator, acetone and MIBK as a solvent and reactant was removed by distillation, to thereby obtain 308 g of a product in which 4-(isopropylamino)-4′-(1,3-dimethylbutylamino) diphenylamine, 4,4′-bis(isopropylamino)diphenylamine and 4,4′-bis(1,3-dimethylbutylamino)diphenylamine were mixed (yield: 95%, as compared to 4,4′-DNDPA).

1. A method for producing 4,4′-bis(alkylamino)diphenylamine, comprising the steps of:
1) reacting urea with nitrobenzene in the presence of a polar organic solvent and a base to prepare 4,4′-dinitrodiphenylamine; and
2) subjecting 4,4′-dinitrodiphenylamine prepared in step 1) and a ketone to hydrogenation in the presence of a hydrogenation catalyst.

2. The method according to claim 1, wherein the polar organic solvent is dimethylsulfoxide, dimethylformamide or N-methylpyrrolidone.

3. The method according to claim 1, wherein the base is an akali metal base an akaline earth metal base or an organic amine base.

4. The method according to claim 1, wherein the urea and nitrobenzene are used in a molar ratio ranging from 1:1 to 1:16.

5. The method according to claim 1, wherein the urea and base are used in a molar ratio ranging from 1:1 to 1:20.

6. The method according to claim 1, wherein the polar organic solvent is used in the amount of 1 to 50 parts by volume based on 1 part by volume of urea and nitrobenzene.

7. The method according to claim 1, wherein the reaction of step 1) is carried out at a temperature ranging from normal temperature to 100° C.

8. The method according to claim 1, wherein the reaction of step 1) is carried out in the presence of oxygen, air or nitrogen.

9. The method according to claim 1, wherein the ketone is alkylketone or cycloalkylketone.

10. The method according to claim 1, wherein the hydrogenation catalyst is precious metals, a reduction catalyst or a carbon supported precious metal catalyst.

11. The method according to claim 1, wherein the hydrogenation of step 2) is carried out at a temperature ranging from 50 to 200° C. and at a hydrogen pressure ranging from 300 to 1000 psi.

12. The method according to claim 1, wherein the ketone and 4,4′-dinitrodiphenylamine are used in a molar ration ranging from 1:3 to 1:10.
